(19) United States
(12) Patent Application Publication
(10) Pub. No.: US 2020/0246520 A1
(43) Pub. Date: Aug. 6, 2020

(54) MASSAGING DEVICE, PROVIDED WITH COOLING AND HEATING SYSTEM, FOR TREATING MUSCLE PAIN AND INFLAMMATION

(71) Applicant: Tae Soun CHANG, Seoul (KR)

(72) Inventor: Tae Soun CHANG, Seoul (KR)

(21) Appl. No.: 16/473,814

(22) PCT Filed: Aug. 17, 2017
(86) PCT No.: PCT/KR2017/008932

§ 371 Date: Jun. 26, 2019

(30) Foreign Application Priority Data
Aug. 18, 2016 (KR) 10-2016-0105022

(51) Int. Cl. A61M 1/08; A61H 9/00

(52) U.S. Cl. CPC A61M 1/08; A61H 9/0057; A61H 9/0007; A61M 2205/3606; A61M 2205/3673; A61M 2205/366; A61H 2201/5082; A61M 2205/3337; A61H 2201/0207; A61H 2201/0214; A61H 2201/0285; A61H 2201/0242; A61M 2205/3368

(57) ABSTRACT

Provided is a massaging device including: a housing; an adsorption unit mounted in the housing and adsorbed on the skin so as to be moved along the skin; and a cooling and heating system installed in the adsorption unit to cool or heat the adsorption unit thereby allowing cold and hot thermal energies to directly penetrate into the skin through the adsorption unit contacting the skin. Further, the cold and hot thermal energies generated in the cooling and heating system can act directly on the skin through the adsorption unit, so that the cold and hot thermal energies penetrate deeply into the skin, thereby removing the waste materials, harmful substances and fatigue substances accumulated in the skin.

### TECHNICAL FIELD

The present invention relates to a massaging device capable of removing skin wastes and fatigue substances as well as relieving muscle pain by providing a cooling and heating system capable of simultaneously providing hot and cold thermal energies to the skin while shrinking and relaxing the skin.

### BACKGROUND ART

In general, a therapeutic method using a cupping device is an herbal medicine therapy used for vacuum blood purification therapy, blood purification and improvement of the physical constitution. In the case of the therapeutic method using a cupping device, a cup is put upside down on an affected part to be treated, and air in the cup is taken out to create a vacuum state therein by using a suction device capable of sucking in air. As a result, a part of the skin of an operation site is sucked into the inside of the cup, and thus the muscle is loosened and the nerve is stabilized. In addition, the pores of the skin are opened, and thus the waste materials and other toxins in the human body that are present in the capillary are leaked through the pores thereof to keep blood to smoothly flow in the blood vessels, and also to extract blood or pus.

In the case of the cupping device, a cup-shaped vessel is provided and connected to a separate vacuum suction device to then be made to come in contact with the human skin and operate the suction device, to thereby lower the pressure inside the cupping device. Alternatively, only a cup is provided and is made to come in contact with the skin of the human body, to then cauterize the skin with moxa inside the cup or connect the cup with a manual pump, so that the air inside the cup is manually pulled out and the inside of the cup is changed to a vacuum. By doing so, the cupping therapy is applied to the human body.

However, such a cupping device performs a simple cupping therapy function in which blood is taken out from the body by using a simple negative pressure, and has the difficulty in controlling the suction pressure, thereby imposing a strain on the skin tissue. As a result, it is difficult to apply the cupping device to a skin care.

In order to solve such a problem, as disclosed in Korean Patent Registration Publication No. 10-0856761 (published on Aug. 29, 2008), a conventional skin massaging device includes: a vacuum source; and a cupping device including a plurality of cups the inside of which a negative pressure is generated by the vacuum source, and of which lower end is inwardly formed in a stepped manner. The cupping device includes: valve units each which forms a flow path when one end of the valve unit is connected to the vacuum source and a negative pressure is applied to the valve unit by the vacuum source; and the plurality of cups overlapped with each other while maintaining a gap therebetween so that a negative pressure is applied to the inside of each of the valve units when the other end of each of the valve units is connected to the upper end of each of the plurality of cups so as to communicate with each of the plurality of cups and the flow path is formed in each of the valve units by the vacuum source.

However, since the conventional skin massaging device merely performs skin massage only by negative pressure, and has no system for providing cold and hot thermal energies to the skin, it is difficult to treat muscle pain and inflammation.

### DISCLOSURE

### Technical Problem

Accordingly, it is an object of the present invention to provide a massaging device for treating muscle pain and inflammation with a cooling and heating system capable of simultaneously providing cold and hot thermal energies to the skin, thereby improving massage performance.

It is another object of the present invention to provide a massaging device for treating muscle pain and inflammation, which has a cooling and heating system capable of enhancing skin contraction and relaxation performance by alternately applying negative pressure and positive pressure to an adsorption unit.

It is still another object of the present invention to provide a massaging device for treating muscle pain and inflammation, which has a cooling and heating system capable of removing waste products, harmful substances and fatigue substances accumulated in the skin, in which cold and hot thermal energies generated from the cooling and heating system are directly acted on the skin through an adsorption unit to thereby make the cold and hot thermal energies penetrate deep into the skin.

### Technical Solution

In order to attain the above object, according to an aspect of the present invention, there is provided a massaging device including: a housing; an adsorption unit mounted in the housing and adsorbed on the skin so as to be moved along the skin; and a cooling and heating system installed in the adsorption unit to cool or heat the adsorption unit thereby allowing cold and hot thermal energies to directly penetrate into the skin through the adsorption unit contacting the skin.

The adsorption unit includes: an air passage portion provided with a vacuum pressure in the center thereof; a seating portion on which the cooling and heating system is seated on the upper surface thereof, and an adsorption cup which is adsorbed on the skin on the lower side thereof. The adsorption cup includes a first adsorption cup disposed outside and a second adsorption cup formed inside the first adsorption cup, wherein the height of the second adsorption cup is lower by a height (H) than the height of the first adsorption cup.

The cooling and heating system includes: a Peltier element disposed in the seating portion and providing hot and cold thermal energies the adsorption unit; and a cooling device for cooling the heat generated in the Peltier element during the cooling operation of the Peltier element. The cooling device is operated when the Peltier element provides a cold thermal energy to the adsorption unit to cool a heat generating portion of the Peltier element, and is stopped to operate when the Peltier element provides a hot thermal energy to the adsorption unit, to allow the heat generated from the heat generating portion of the Peltier element to be transmitted to a heat absorbing portion.

The cooling device includes: a cooling water chamber disposed in contact with the heat generating portion of the Peltier element and filled with cooling water; a radiator connected to an exhaust pipe of the cooling water chamber by a hose, and cooling the heated cooling water discharged from the cooling water chamber; a cooling water tank connected to the radiator and the cooling water chamber and storing the cooling water; and a cooling water pump installed in the cooling water tank and pumping the cooling water to circulate.

The cooling and heating system includes: a Peltier element disposed in a seating portion of the adsorption unit to provide a cold thermal energy to the adsorption unit; a heating element disposed in the circumferential direction at an edge of the seating portion of the adsorption unit to provide a hot thermal energy to the adsorption unit; and a cooling device disposed on the upper surface of the Peltier element to cool the Peltier element.

The cooling and heating system further includes: a temperature sensor installed in the adsorption unit and measuring the temperature of the hot and cold thermal energies generated in the cooling and heating system and transferred to the adsorption unit; and a control unit for controlling the heat generated from the cooling and heating system according to a signal applied from the temperature sensor.

The massaging device further includes: a vacuum pump for generating a negative pressure to be supplied to the adsorption unit; a pulse solenoid valve disposed between the vacuum pump and the adsorption unit for repeatedly providing a negative pressure and a positive pressure to the adsorption unit; a compressor connected to the pulse solenoid valve and generating a positive pressure when a negative pressure of the vacuum pump is provided; and a vacuum pressure control valve for adjusting an air pressure of the adsorption unit by steps according to a user's selection.

### Advantageous Effects

As described above, the massaging device according to an embodiment of the present invention is provided with a cooling and heating system to simultaneously provide cold and hot thermal energies to the skin, thereby improving the massage performance.

In addition, by allowing a negative pressure and a positive pressure to be alternately applied to the adsorption unit, skin shrinkage and relaxation performance can be improved.

Further, the cold and hot thermal energies generated in the cooling and heating system can act directly on the skin through the adsorption unit, so that the cold and hot thermal energies penetrate deeply into the skin, thereby removing the waste materials, harmful substances and fatigue substances accumulated in the skin.

### BRIEF DESCRIPTION OF THE DRAWINGS

FIG. 1 is a configuration block diagram of a massaging device according to an embodiment of the present invention.

FIG. 2 is a cross-sectional view of a massaging device according to a first embodiment of the present invention.

FIG. 3 is an exploded perspective view of a massaging device according to an embodiment of the present invention.

FIG. 4 is a partially enlarged cross-sectional view of a massaging device according to an embodiment of the present invention

FIG. 5 is a block diagram of a control unit in a massaging device according to an embodiment of the present invention.

FIG. 6 is a partial cross-sectional view of a massaging device according to another embodiment of the present invention.

### BEST MODE

Hereinafter, embodiments of the present invention will be described in detail with reference to the accompanying drawings. The sizes and shapes of the components shown in the drawings may be exaggerated for clarity and convenience. In addition, terms defined in consideration of the configuration and operation of the present invention may vary depending on the intention or custom of the user, the operator, and the like. Definitions of these terms should be based on the content of this specification.

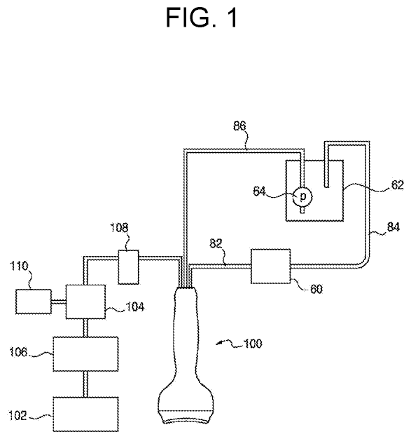

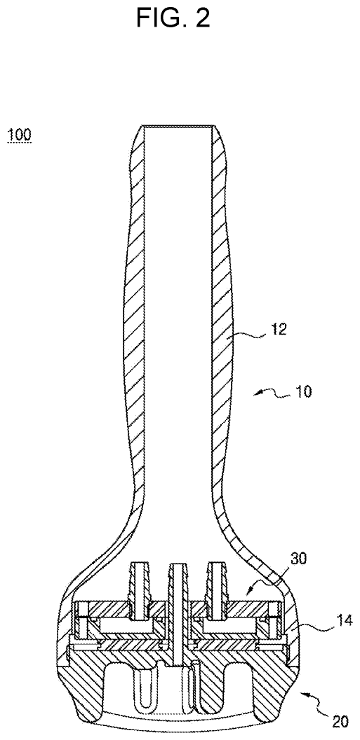

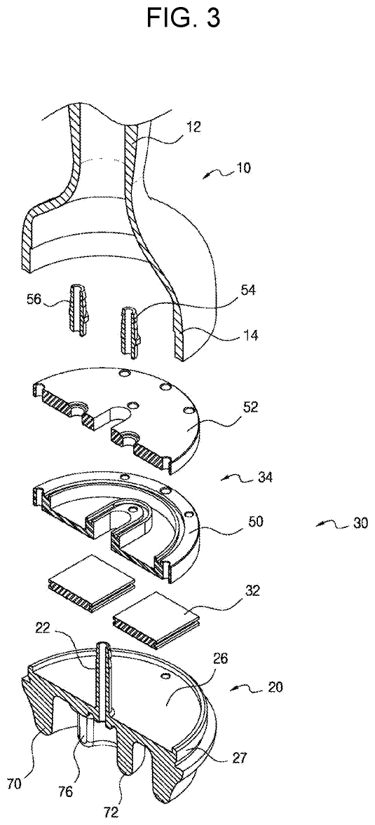

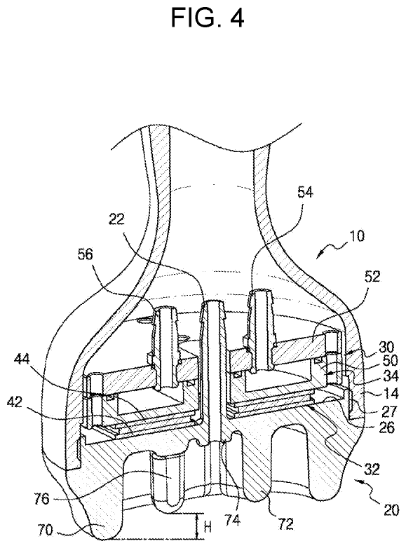

FIG. 1 is a configuration block diagram of a massaging device according to an embodiment of the present invention. FIG. 2 is a cross-sectional view of a massaging device according to a first embodiment of the present invention. FIG. 3 is an exploded perspective view of a massaging device according to an embodiment of the present invention. FIG. 4 is a partially enlarged cross-sectional view of a massaging device according to an embodiment of the present invention.

A massaging device 100 includes: a housing 10 provided with a grip portion 12; an adsorption unit 20 mounted in the housing 10 and adsorbed on the skin so as to be moved along the skin; and a cooling and heating system 30 installed in the adsorption unit 20 to provide cold and hot thermal energies to the adsorption unit 20.

The grip portion 12 for gripping by hand is formed on the upper side of the housing 10 and an adsorption unit fixing portion 14 to which the adsorption unit 20 is fixed is formed on the lower side of the housing 10.

The adsorption unit 20 includes: an air passage portion 22 provided with a vacuum pressure in the center thereof; a seating portion 26 on which the cooling and heating system 30 is seated on the upper surface thereof; and a plurality of adsorption cups 70 and 72 which are adsorbed on the skin on the lower side thereof. A stepped portion 27 is formed on the outer surface of the adsorption unit 20 and is coupled to the adsorption unit fixing portion 14 of the housing 10.

The adsorption cups 70 and 72 include a first adsorption cup 70 disposed outside and a second adsorption cup 72 formed inside the first adsorption cup 70.

The height of the second adsorption cup 72 is formed to be lower than the height of the first adsorption cup 70 by a predetermined height H, for example. When the skin is sucked into the first adsorption cup 70, the second adsorption cup 72 having a height lower than that of the first adsorption cup 70 presses the skin, so that the skin becomes wave-shaped to improve the massage performance of the skin.

The second adsorption cup 72 includes an air guide groove 76 for guiding a negative pressure or a positive pressure provided through the air passage portion 22 into the first adsorption cup 70.

A recessed cream inflow preventing portion 74 for preventing cream or moisture from flowing into an air path 46 is formed on the inner upper surface of the second adsorption cup 72. When the skin is massaged with the adsorption unit 20, the cream is applied to the skin and massage is performed while moving along the skin. Here, when the cream flows into the air passage portion 22, the air passage portion 22 may be clogged or damage may be caused to the vacuum pump or the like.

The cream inflow preventing portion 74 prevents the cream from flowing into the air passage portion 22, thereby preventing the hose from being clogged by the cream or from damaging other components by the cream.

Specifically, when the cream, moisture, or the like is sucked into the second adsorption cup 72, the sucked cream, moisture, or the like flows in along the inner wall of the second adsorption cup 72. Here, the cream or the moisture is blocked by the cream inflow preventing portion 74 of a recessed form and is prevented from flowing into the air passage portion 22.

The adsorption unit 20 is formed of a thermally conductive metal material having excellent thermal conductivity so as to transmit heat generated from the cooling and heating system 30 to the skin. That is, the adsorption unit 20 may be formed of copper, aluminum, or an alloy thereof.

The cooling and heating system 30 includes: a Peltier element 32 disposed in the seating portion 26 for providing hot and cold thermal energies to the adsorption unit 20; and a cooling device 34 for cooling the heat generated from the Peltier element 32 during the cooling operation of the Peltier element 32.

The Peltier element 32 is provided with a heat absorbing portion 42 that absorbs heat when power is applied thereto and a heat generating portion 44 that radiates heat. The heat absorbing portion 42 is disposed in the seating portion 26 and the heat generating portion 44 is provided with a cooling device 34 for cooling the heat generated in the heat generating portion 44.

The cooling device 34 includes: a cooling water chamber 50 which is disposed in contact with the heat generating portion 44 of the Peltier element 32 and is filled with cooling water; a sealing lid 52 which sealably covers the open top surface of the cooling water chamber 50; a supply pipe 54 mounted on the sealing lid 52 and supplying cooling water to the cooling water chamber 50; and an exhaust pipe 56 mounted on the sealing lid 52 and discharging cooling water from the cooling water chamber 50.

According to an embodiment of the present invention, the cooling device is provided to cool a Peltier element by a water cooling method and includes: a radiator 60 connected to the exhaust pipe 56 through a first cooling water line 82 to cool the heated cooling water discharged from the cooling water chamber 50; a cooling water tank 62 connected to the radiator 60 through a second cooling water line 84 and connected to the supply pipe 54 through a third cooling water line 86 and storing cooling water therein; and a cooling water pump 64 installed in the cooling water tank 62 to pump the cooling water to circulate.

Here, the cooling water is characterized in that an antifreeze and distilled water are mixed in a ratio of 5:5, in order to improve cooling performance.

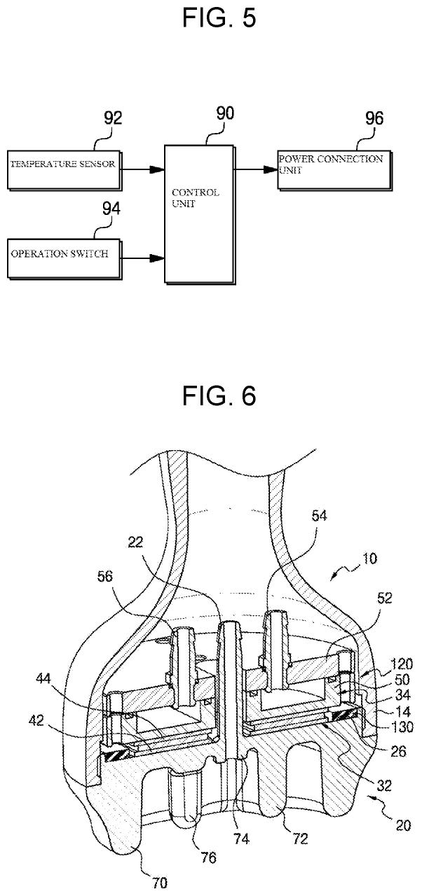

In addition, as shown in FIG. 5, the cooling and heating system 30 further includes: a temperature sensor 92 installed in the adsorption unit 20 and measuring the temperature of the hot and cold thermal energies generated in the cooling and heating system 30 and transferred to the adsorption unit 20; and a control unit 90 for controlling the temperature of the heat generated from the cooling and heating system 30 according to a signal applied from the temperature sensor 92.

That is, when the heat generated from the cooling and heating system 30 in accordance with a signal applied from the temperature sensor 92 is equal to or higher than a predetermined temperature, the control unit 90 controls a power connection unit 96 so as not to apply power to the cooling and heating system 30. Meanwhile, when the heat generated from the cooling and heating system 30 in accordance with a signal applied from the temperature sensor 92 is equal to or lower than a predetermined temperature, the control unit 90 controls the power connection unit 96 so as to apply power to the cooling and heating system 30. Accordingly, the control unit 90 controls the cooling and heating system 30 to generate a constant heat at all times.

When a user operates an operation switch 94 to set a temperature of the cooling and heating system 30, the control unit 90 controls the cooling and heating system 30 according to the temperature set in the operation switch 94.

When the heat generated from the cooling and heating system 30 is equal to or higher than the set temperature, the adsorption unit 20 is provided with a bimetal (not shown) for preventing a user from being burnt by mechanically shutting off the power applied to the cooling and heating system 30.

The cooling and heating system 30 constructed as above will be described below. First, a cooling water pump 64 and a radiator 60 are operated during the cooling and heating operation. The heat generated in the heat generating portion 44 of the Peltier element 32 is cooled by the cooling device 34 and the heat absorbing function of the adsorption unit 20 is performed by the heat absorbing portion 42 of the Peltier element 32, such that the adsorption unit 20 is cooled.

During the heating operation, the operation of the cooling water pump 64 and the radiator 60 is stopped. The heat generated by the heat generating portion 44 of the Peltier element 32 is not dissipated and is transmitted to the heat absorbing portion 42 of the Peltier element 32, and the heat absorbing portion 42 is heated by the heat of the heat generating portion 44, such that the overall temperature of the Peltier element 32 is raised to provide heat of a hot thermal energy to the adsorption unit 20.

As described above, the adsorption unit 20 applied to the embodiment of the present invention is formed of a metal material having excellent thermal conductivity, so that the hot and cold thermal energies generated in the cooling and heating system 30 are directly transferred to the skin through the adsorption unit 20 adsorbed on the skin without passing through the air. As a result, the hot and cold thermal energies generated in the cooling and heating system 30 can penetrate deeply into the skin, so that waste, fatigue and harmful substances can be removed.

In some embodiments, the massaging device further includes: a vacuum pump 102 for generating a negative pressure to be supplied to the adsorption unit 20; a pulse solenoid valve 104 disposed between the vacuum pump 102 and the adsorption unit 20 for repeatedly providing a negative pressure and a positive pressure to the adsorption unit 20; a compressor 110 connected to the pulse solenoid valve 104 and generating a positive pressure when a negative pressure of the vacuum pump 102 is provided; and a vacuum pressure control valve 106 for adjusting an air pressure of the adsorption unit 20 by steps according to a user's selection.

In addition, the vacuum pressure line is equipped with a filter 108 to block the introduction of various foreign substances into the vacuum pump or the like from the adsorption unit 20.

The pulse solenoid valve 104 plays a role of repeatedly transmitting a negative pressure and a positive pressure to the adsorption unit 20 by using a negative pressure generated in the vacuum pump 102. That is, when the pulse solenoid valve 104 is opened, a negative pressure generated in the vacuum pump 102 is supplied to the adsorption unit 20 to contract the skin. When the pulse solenoid valve 104 is closed, a positive pressure generated in the vacuum pump 102 is supplied to the adsorption unit 20 to relax the skin.

The compressor 110 is connected to the pulse solenoid valve 104 and is operated by a negative pressure generated in the vacuum pump 102 to blow out the external air to the adsorption unit 20.

The vacuum pressure control valve 106 regulates the vacuum pressure to be supplied to the adsorption unit 20 according to a user's selection.

FIG. 6 is a partial cross-sectional view of a massaging device according to another embodiment of the present invention.

The massaging device according to another embodiment is the same as the massaging device described in the above embodiment, except that a cooling and heating system 120 has a different structure from the cooling and heating system 30 of the previous embodiment.

The cooling and heating system 120 according to another embodiment includes: a Peltier element 32 disposed in a seating portion 26 of the adsorption unit 20 to provide a cold thermal energy to the adsorption unit 20; a heat generating element 130 disposed in the circumferential direction at an edge of the seating portion 26 of the adsorption unit 20 to provide a hot thermal energy to the adsorption unit 20; and a cooling device 34 disposed on the upper surface of the Peltier element 32 to cool the Peltier element 32.

The cooling and heating system 120 according to another embodiment applies power to the Peltier element 32 and operates the cooling device 34 when a cold thermal energy is provided for the adsorption unit 20, such that a heat absorbing function of the adsorption unit 20 is performed in a heat absorbing portion 42 of the Peltier element 32 to cool the adsorption unit 20.

Meanwhile, when the hot thermal energy is supplied to the adsorption unit 20, power is applied to the heat generation element 130 to generate heat in the heat generation element 130, and this heat is transferred to the adsorption unit 20, to heat the adsorption unit 20.

While the present invention has been particularly shown and described with reference to exemplary embodiments thereof, by way of illustration and example only, it is clearly understood that the present invention is not to be construed as limiting the present invention, and various changes and modifications may be made by those skilled in the art within the protective scope of the invention without departing off the spirit of the present invention.

### INDUSTRIAL APPLICABILITY

The present invention is a massaging device capable of treating muscular pain and inflammation by allowing a negative pressure and static pressure to be alternately applied to the skin, and capable of providing hot and cold thermal massaging effects to the skin because both cold and hot thermal energies can be provided to the skin.

What is claimed is:

1. A massaging device comprising:
a housing;
an adsorption unit mounted in the housing and adsorbed on the skin so as to be moved along the skin; and
a cooling and heating system installed in the adsorption unit to cool or heat the adsorption unit thereby allowing cold and hot thermal energies to directly penetrate into the skin through the adsorption unit contacting the skin.

2. The massaging device of claim 1, wherein the adsorption unit comprises:
an air passage portion provided with a vacuum pressure in the center thereof;
a seating portion on which the cooling and heating system is seated on the upper surface thereof; and
an adsorption cup which is adsorbed on the skin on the lower side thereof.

3. The massaging device of claim 2, wherein the adsorption cup comprises:
a first adsorption cup disposed outside; and
a second adsorption cup formed inside the first adsorption cup, wherein the height of the second adsorption cup is lower by a height (H) than the height of the first adsorption cup.

4. The massaging device of claim 2, wherein the cooling and heating system comprises:
a Peltier element disposed in the seating portion and providing hot and cold thermal energies the adsorption unit; and
a cooling device for cooling the heat generated in the Peltier element during the cooling operation of the Peltier element,
wherein the cooling device is operated when the Peltier element provides a cold thermal energy to the adsorption unit to cool a heat generating portion of the Peltier element, and is stopped to operate when the Peltier element provides a hot thermal energy to the adsorption unit, to allow the heat generated from the heat generating portion of the Peltier element to be transmitted to a heat absorbing portion.

5. The massaging device of claim 4, wherein the cooling device comprises:
a cooling water chamber disposed in contact with the heat generating portion of the Peltier element and filled with cooling water;
a radiator connected to an exhaust pipe of the cooling water chamber by a hose, and cooling the heated cooling water discharged from the cooling water chamber;
a cooling water tank connected to the radiator and the cooling water chamber and storing the cooling water; and
a cooling water pump installed in the cooling water tank and pumping the cooling water to circulate.

6. The massaging device of claim 5, wherein the cooling water is characterized in that an antifreeze and distilled water are mixed in a ratio of 5:5.

7. The massaging device of claim 2, wherein the cooling and heating system comprises:
a Peltier element disposed in a seating portion of the adsorption unit to provide a cold thermal energy to the adsorption unit;
a heating element disposed in the circumferential direction at an edge of the seating portion of the adsorption unit to provide a hot thermal energy to the adsorption unit; and
a cooling device disposed on the upper surface of the Peltier element to cool the Peltier element.

8. The massaging device of claim 1, wherein the cooling and heating system comprises:
a temperature sensor installed in the adsorption unit and measuring the temperature of the hot and cold thermal energies generated in the cooling and heating system and transferred to the adsorption unit; and
a control unit for controlling the heat generated from the cooling and heating system according to a signal applied from the temperature sensor.

9. The massaging device of claim 1, further comprising:
a vacuum pump for generating a negative pressure to be supplied to the adsorption unit;
a pulse solenoid valve disposed between the vacuum pump and the adsorption unit for repeatedly providing a negative pressure and a positive pressure to the adsorption unit;
a compressor connected to the pulse solenoid valve and generating a positive pressure when a negative pressure of the vacuum pump is provided; and
a vacuum pressure control valve for adjusting an air pressure of the adsorption unit by steps according to a user's selection.
